(19) United States
(12) Patent Application Publication
(10) Pub. No.: US 2025/0161715 A1
(43) Pub. Date: May 22, 2025

(54) MOTION COMPENSATION DURING RADIATION TREATMENT

(71) Applicant: Varian Medical Systems, Inc., Palo Alto, CA (US)

(72) Inventors: Pascal Paysan, Basel (CH); Stefan Scheib, Wadenswil (CH); Leigh Johnson, Chicago, IL (US)

(21) Appl. No.: 18/511,250

(22) Filed: Nov. 16, 2023

(51) Int. Cl. A61N 5/10; G16H 20/40

(52) U.S. Cl. CPC A61N 5/1037; A61N 5/1039; G16H 20/40

(57) ABSTRACT

A control circuit can access multi-dimensional information for a particular patient and then automatically determine a supplemental boundary for at least one portion of the particular patient (such as a treatment target and/or one or more organs-at-risk) as a function, at least in part, of the multi-dimensional information. The latter may comprise determining a margin that is added to a boundary of the at least one portion of the particular patient. The control circuit can then, for example, determine a planning treatment volume as a function, at least in part, of that supplemental boundary. These teachings will then permit, for example, optimizing a radiation-based therapeutic treatment plan for the particular patient as a function of various dimensions of movement as derived, at least in part, from the aforementioned multi-dimensional information.

### TECHNICAL FIELD

These teachings relate generally to treating a patient's planning target volume with energy pursuant to an energy-based treatment plan and more particularly to optimizing an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

A radiation treatment plan typically comprises specified values for each of a variety of treatment-platform parameters during each of a plurality of sequential fields. Treatment plans for radiation treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

One challenge to confidently planning and administering radiation to a patient is motion within the treatment area. Some parts of the human body, such as the heart, parts of the body associated with breathing, and parts of the body associated with gastric activities can and do move over time. Such movements can move portions of both targeted and protected areas to different locations. As a result, under or overdosing of the target and overdosing of protected areas can occur.

### BRIEF DESCRIPTION OF THE DRAWINGS

The above needs are at least partially met through provision of the apparatus and method for motion compensation during radiation treatment described in the following detailed description, particularly when studied in conjunction with the drawings, wherein:

FIG. 1 comprises a block diagram as configured in accordance with various embodiments of these teachings;

FIG. 2 comprises a chart as configured in accordance with various embodiments of these teachings;

FIG. 3 comprises a chart as configured in accordance with various embodiments of these teachings;

FIG. 4 comprises a flow diagram as configured in accordance with various embodiments of these teachings; and

FIG. 5 comprises a flow diagram as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, pursuant to these various embodiments a control circuit can access multi-dimensional information for a particular patient and then automatically determine a supplemental boundary for at least one portion of the particular patient (such as a treatment target and/or one or more organs-at-risk) as a function, at least in part, of the multi-dimensional information. The latter may comprise, for example, determining a margin that is added to a boundary of the at least one portion of the particular patient. So configured, the control circuit can then, for example, determine a planning treatment volume as a function, at least in part, of that supplemental boundary. These teachings will then permit, for example, optimizing a radiation-based therapeutic treatment plan for the particular patient as a function of various dimensions of movement as derived, at least in part, from the aforementioned multi-dimensional information.

The aforementioned multi-dimensional information can include, for example, the three Cartesian orientations and a fourth, fifth, or more dimension that can refer to any of a variety of patient-centric motion-based references. Examples include, but are not limited to, cardiac-based imagery, respiratory-based imagery, and cyclic gastric motion-based imagery for the particular patient.

As one example, the multi-dimensional information can comprise four or five-dimensional information for the particular patient. In such a case, the fourth and fifth dimensions can refer to different patient-centric motion-based references. Similarly, these teachings will accommodate using six or more dimensions, where, for example, the fourth, fifth, and sixth dimensions correspond to cardiac-based imagery, respiratory-based imagery, and cyclic gastric motion-based imagery for the particular patient.

These teachings are highly flexible in practice and will accommodate any of a variety of modifications and/or supplemental features.

As one example in the foregoing regards, these teachings will accommodate presenting the aforementioned motion-based imagery to a user and then providing that user, via a user interface, with an opportunity to selectively modify the playback of movement of one motion-based imagery (such as, for example, cardiac-based imagery) separately from another motion-based imagery (such as, for example, respiratory-based imagery).

As another example in the foregoing regards, these teachings will accommodate generating a motion model for the particular patient as a function, at least in part, of the aforementioned multi-dimensional information for the particular patient. Such a motion model can then serve as a basis, for example, for assessing efficacy for each of a plurality of different therapeutic treatment modalities for the particular patient.

By one approach, these teachings will support accessing supplemental multi-dimensional information for the particular patient at the time of treatment and then updating the aforementioned motion model as a function, at least in part, of that supplemental multi-dimensional information and/or validating the motion model as a function, at least in part, of that supplemental multi-dimensional information.

As yet another example of the flexibility of these teachings, by one approach these teachings will accommodate reconstructing an absorbed dose administered during a therapeutic radiation treatment session as a function, at least in part, of the aforementioned multi-dimensional information for the particular patient and the aforementioned motion model for the particular patient that was generated as a function, at least in part, of the multi-dimensional information for the particular patient.

So configured, one or more motion modalities for a particular patient can be better understood and leveraged to yield a radiation treatment plan and corresponding treatment that is likely to produce better overall results for the patient. These better results can include more precise (and/or consistent) dosing of a treatment target and/or more effective protection for non-targeted areas from radiation.

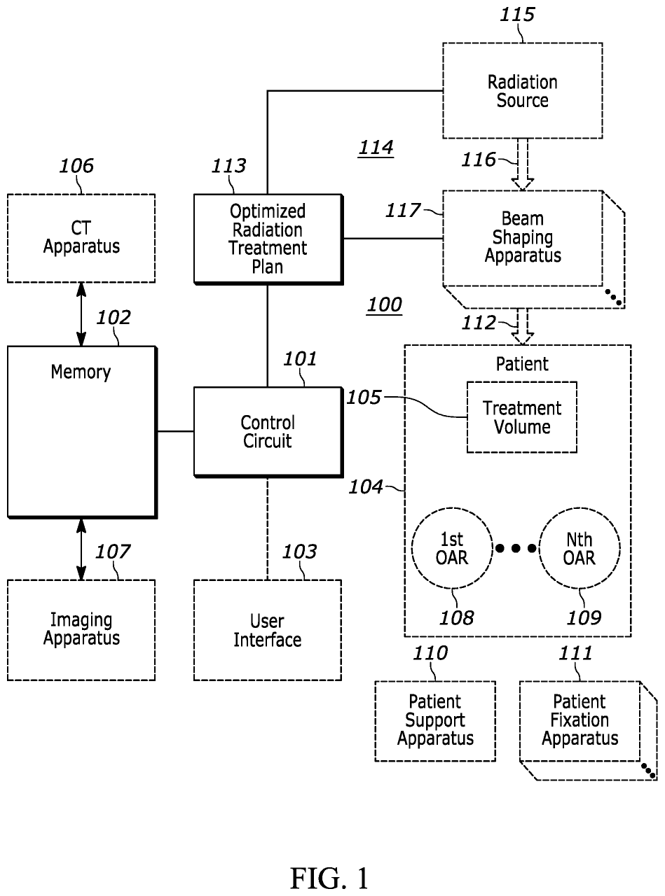

These and other benefits may become clearer upon making a 106 review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101).

In addition to information such as the image information described herein, optimization information for a particular patient, and information regarding a particular radiation treatment platform as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless platforms, are well understood in the art and require no particular elaboration here.

By one approach, a computed tomography apparatus 106 and/or other imaging apparatus 107 (such as positron emission tomography and/or magnetic resonance imaging (including both functional and quantitative approaches) as are known in the art can source some or all of any desired patient-related imaging information. By one approach, these teachings will support using essentially any imaging modality that may be available on the treatment delivery device to generate day-of-treatment images to update and validate, for example, a motion model. Examples of useful image modalities include, but are not limited to, X-ray projection imaging, X-ray fluoroscopy imaging, CBCT, polychromatic CBCT, dual Energy CBCT, 4D-CBCT, 5D-CBCT, and even, for example, such things as cardiac-specific imaging and planning tools.

In this illustrative example the control circuit 101 is configured to ultimately output an optimized energy-based treatment plan (such as, for example, an optimized radiation treatment plan 113). This energy-based treatment plan typically comprises specified values for each of a variety of treatment-platform parameters during each of a plurality of sequential exposure fields. In this case the energy-based treatment plan is generated through an optimization process, examples of which are provided further herein.

By one approach the control circuit 101 can operably couple to an energy-based treatment platform 114 that is configured to deliver therapeutic energy 112 to a corresponding patient 104 having at least one treatment volume 105 and also one or more organs-at-risk (represented in FIG. 1 by a first through an Nth organ-at-risk 108 and 109) in accordance with the optimized energy-based treatment plan 113. These teachings are generally applicable for use with any of a wide variety of energy-based treatment platforms/apparatuses. In a typical application setting the energy-based treatment platform 114 will include an energy source such as a radiation source 115 of ionizing radiation 116.

By one approach this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment platform 114 may also include one or more support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction and also including rotational movement) during an energy-based treatment session by the control circuit 101. As the foregoing elements and systems are well understood in the art, further elaboration in these regards is not provided here except where otherwise relevant to the description.

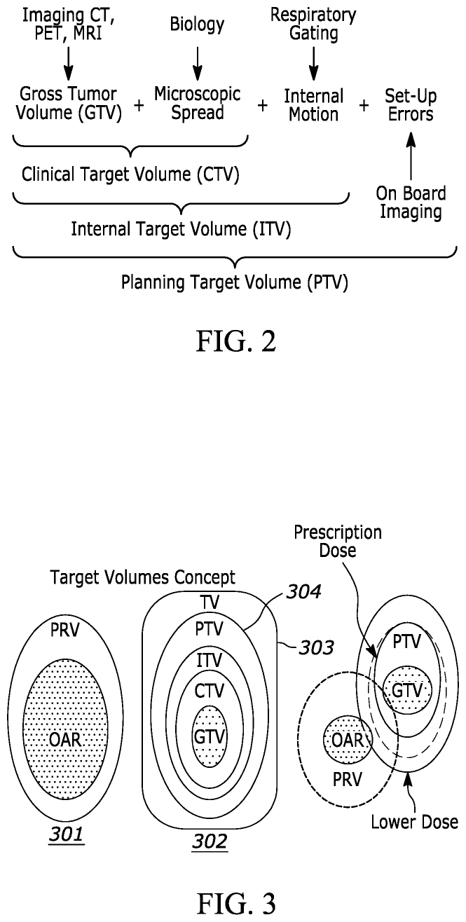

Referring to FIGS. 2 and 3, it may be helpful to describe and explain certain terms, acronyms, and concepts that can be relevant to the present teachings.

A patient's gross tumor volume (GTV) can be determined from patient images (such as computed tomography images, positron emission tomography imagers, and/or magnetic resonance imaging images). That gross tumor volume can be coupled with microscopic spread information to determine a clinical target volume (CTV). That microscopic spread information can be based, for example, upon the known biological record for a particular type of tumor in a particular type of tissue.

An internal target volume (ITV) can then be based upon the clinical target volume in conjunction with patient internal motion information (derived, for example, from observations of the respiratory system). A planning target volume (PTV) can then be based upon the foregoing internal target volume and an additional margin or safety/error that is based upon, for example, potential and/or observed set-up errors.

As shown in FIG. 3 at reference numeral 301, a planning organ-at-risk outer boundary can be provided for a given organ-at-risk that takes into account motion information that can influence/determine a volume within which the organ-at-risk may exist during the treatment session. Reference numeral 302 schematically presents the boundaries/margins discussed above for the patient's targeted volume. A corresponding target volume (TV) is also depicted and will be described below in more detail.

By one approach, one can link the right most part of FIG. 3 to the optimized treatment plan as a function of the motion analyses that is based on multi-dimensional imaging and a motion model that is built for the specific patient. Based on the foregoing, these teachings will support determining the patient's individual planning organ-at-risk volumes and patient treatment volumes and also the plan alternatives that will potentially differ regarding the shaped isodose lines with respect to the organs-at-risk. The sketched-out prescription and lower isodose lines may be one of several plan realizations.

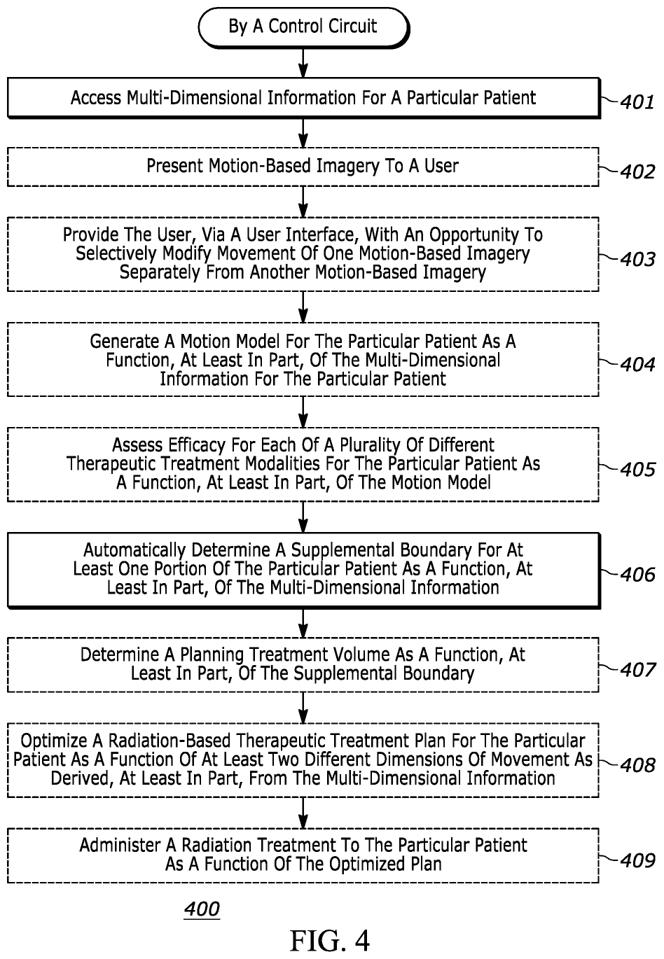

Referring now to FIG. 4, a process 400 that can be carried out, for example, in conjunction with the above-described application setting (and more particularly via the aforementioned control circuit 101) will be described. Generally speaking, this process 400 serves to facilitate generating an optimized radiation treatment plan 113 to thereby facilitate treating a particular patient with therapeutic radiation using a particular radiation treatment platform per that optimized radiation treatment plan. More particularly, this process 400 can serve to generally compensate for motion during a therapeutic treatment session for that particular patient.

At block 401, this process 400 provides for accessing multi-dimensional information for the particular patient. As used herein, the expression “multi-dimensional” will be understood to refer to at least the three standard Cartesian dimensions plus one or more additional motion-based dimensions. Examples of motion-based dimensions include, but are not limited to, the patient's cardiac-based activity, respiratory-based activity, and/or cyclic gastric-based activity.

As one illustrative example in these regards, this activity can comprise accessing motion-based imagery comprising one, two, or all three of cardiac-based imagery for the particular patient, respiratory-based imagery for the particular patient, and cyclic gastric motion-based imagery for the particular patient. The foregoing imagery may share a common image-capture modality or may utilize a variety of different image-capture modalities, such as computed tomography, positron emission tomography, and/or magnetic resonance imaging, to note but three examples.

By one optional approach, and as illustrated at optional block 402, this process 400 will accommodate presenting some or all of the aforementioned motion-based imagery to a user (via, for example, the above-described user interface 103). The displayed imagery may be static imagery (including, for example, a single frame of video content) and/or video content. Such images may be displayed with, or without, additional content (such as graphic additions, icons, explanatory or labeling text, and so forth) as desired.

At optional block 403, these teachings will then accommodate providing the user (again via the aforementioned user interface 103) with an opportunity to selectively modify movement of one or more of the display images. The latter may comprise, for example, allowing a user to employ a cursor to select a specific portion or a specific area of the displayed image and then manipulate that portion/area by, for example, moving the cursor and/or selecting an available change via a submenu or the like.

By one approach, the aforementioned modification can comprise slowing down or halting displayed patient movements. The modification may also accommodate switching (abruptly or via a more gradual transition) to a different motion dimension. As one example, the modification may comprise adding breathing motion using a lower amplitude to reflect that a compressing device will be deployed on the patient's belly to help suppress breathing motion amplitude.

By one approach, this opportunity may comprise permitting the user to selectively modify movement of one motion-based image separately from another displayed (or not displayed) motion-based image; in other words, modifying the one motion-based image does not lead to any automatic changes to any other currently-display motion-based image. Using this approach, and as an illustrative example, breathing motion might be modified while cardiac motion is displayed without any concurrent modification.

In lieu of the foregoing, or in combination therewith, at optional block 404 these teachings will accommodate generating a motion model for the particular patient as a function, at least in part, of the multi-dimensional information for the particular patient. (In lieu of this, or in combination with this, this process 400 will accommodate accessing a motion model that is based upon a cohort of other patients, which motion model may then be further modified to include the current particular patient if desired.) These teachings will accommodate employing any of a variety of models including, for example, any of a variety of generative models. Examples of models/algorithms include, but are not limited to, classical linear models, linear regression, principal component analysis (an unsupervised learning technique), and generative neural nets such as variational autoencoders (whose encodings distribution is typically regularised during training) and generative adversarial networks, to note but a few.

Presuming the availability of one or more such motion models, at optional block 405 the motion model(s) can be used to assess efficacy for each of a plurality of different therapeutic treatment modalities for the particular patient as a function, at least in part, of that motion model (or models). For example, one approach to radiation therapy could be compared to another, different approach to radiation therapy by determining a corresponding efficacy for each approach as a function of the motion model (and/or the herein-mentioned mid and mean position information) and then comparing those efficacy determinations to identify an approach that appears likely to be most efficacious.

At block 406, this process 400 provides for automatically determining a supplemental boundary for at least one portion of the particular patient as a function, at least in part, of the multi-dimensional information. The foregoing portion may be, for example, a treatment target or some specified portion thereof and/or an organ-at-risk (or organs-at-risk) or some specified portion thereof. (By one approach, the foregoing portion may comprise some part of a PTV and/or a PRV.)

By one approach, that supplemental boundary may comprise, for example, a margin that is added to an already-established boundary of the at least one portion of the particular patient. Such a margin may be calculated and/or otherwise selected to be sufficient to accommodate a particular extent of movement that can occur with respect to the aforementioned portion of the patient's body.

By one optional approach, and as illustrated at optional block 407, this process 400 will accommodate determining a planning treatment volume as a function, at least in part, of the aforementioned supplemental boundary. This concept is illustrated in the aforementioned FIG. 3, where a margin denoted as a target volume 303 is added to the previously-determined planning target volume. These teachings are flexible in practice and will accommodate using different margin dimensions for different parts of the portion. For example, the margin may be smaller (or even zero) in a direction that does not change during expected patient motion while the margin can be larger in a direction that is more likely to contain a greater range of patient movement. It will be understand that this same approach could be utilized with an organ-at-risk as well.

At optional block 408, this process 400 will accommodate optimizing a radiation-based therapeutic treatment plan for the particular patient as a function of the aforementioned multi-dimensional patient movements. When the multi-dimensional information contains information for two or more different kinds of patient movement, the foregoing can include optimizing the plan as a function of the at least two different dimensions of movement that are derived, at least in part, as a function of the multi-dimensional information.

At optional block 409 the process 400 can then provide for administering a resultant optimized radiation treatment to the particular patient as a function of that optimized plan (using, for example, the aforementioned radiation treatment platform 114).

In the description above, a motion model can serve to help with optimization of a radiation treatment plan for a particular patient. These teachings can also be leveraged at the time of treatment or in the aftermath of a treatment for that particular patient.

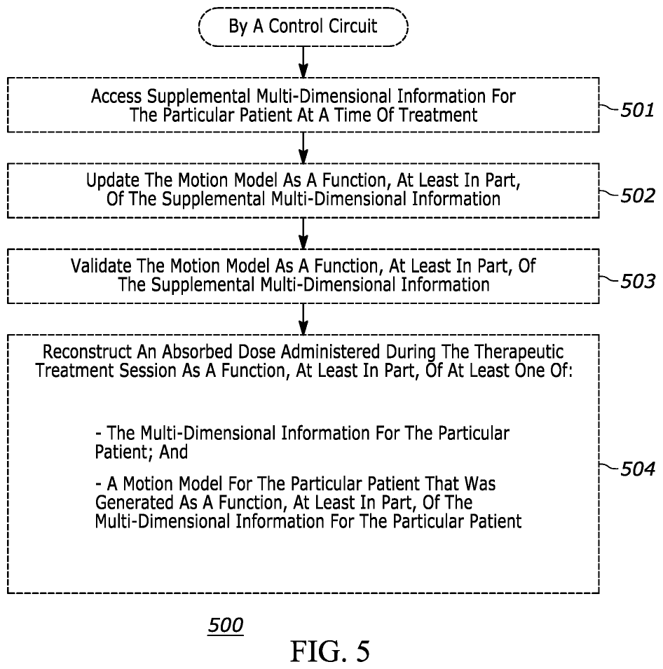

Referring to FIG. 5, at optional block 501, this process 500 will optionally provide for accessing supplemental multi-dimensional information for the particular patient at a time of treatment. As used herein, this reference to “supplemental” will be understood to be multi-dimensional information that is in addition to the originally accessed multi-dimensional information as described above and which also represents a period of time that is at least partially coincident with the treatment window. This supplemental multi-dimensional information can be sourced from the same image sources as originally served, but alternative sources can also be partially or wholly relied upon if desired. The latter can include, for example, on-board imaging capabilities available on the treatment delivery device or devices themselves as mentioned earlier.

That supplemental multi-dimensional information can then be leveraged in any of a variety of ways.

By one approach, and as shown at optional block 502, the aforementioned supplemental multi-dimensional information can be leveraged by updating the aforementioned motion model as a function, at least in part, of that supplemental multi-dimensional information.

By another approach, and as shown at optional block 503, in lieu of the foregoing or in combination therewith, this process 500 will provide for validating the aforementioned motion model as a function, at least in part, of that supplemental multi-dimensional information.

Regardless of whether supplemental multi-dimensional information is developed, and by yet another approach, in lieu of the foregoing or in combination therewith, and as shown at optional block 504, these teachings will allow for reconstructing an absorbed dose administered during the therapeutic treatment session as a function, at least in part, of at least one of the multi-dimensional information for the particular patient and/or a motion model for the particular patient that was generated as a function, at least in part, of the multi-dimensional information for the particular patient.

Further details will now be provided by way of particular application settings and examples. It will be understood that these teachings are not to be viewed as being limited by the specific details of these settings and examples.

In radiation therapy, many techniques have been applied to account for patient motion. Cardiac radio ablation is not yet typically viewed as a broadly established treatment method. Therefore, there is no generally established method to handle target motion in a cardiac radio ablation application setting. In cardiac radio ablation, the treatment target is typically an area of the ventricular wall, which is identified prior to treatment planning with anatomical (4DCT, 4D-MRI) and functional (PET) imaging to locate the ablation target, in addition to the cardiology-specific target localization techniques.

In addition to the heart and, therefore, corresponding target motion, there is breathing motion. These contributing factors typically lead to relatively large motion amplitudes of the target. The latter is often compensated for in radiation therapy by expanding the gross or clinical target volume (GTV, CTV) by that motion amplitude, thereby yielding an internal target volume (ITV). That motion amplitude can be, for example, 10 mm. Adding set-up uncertainties (typically 3-5 mm) to this ITV results in the planning target volume (PTV).

The contribution due to the target motion is, in this case, typically significantly larger compared to the set-up uncertainty. This margin increase, therefore, can lead to a rather large PTV and a corresponding significant dose contribution to non-targeted tissues and organs at risk (OAR) because the entirety of the PTV will, according to the corresponding plan, receive the prescribed therapeutic dose. This adverse effect of high doses to OARs can itself leads to dose reductions in the target to avoid such adverse dosing of sensitive tissues. In cardiac radio ablation, there is already a generally accepted dose of 25 Gy in a single fraction. Limiting the impact of target motion (or reducing the GTV/CTV to the PTV margin) can be very helpful to avoiding either underdosing of the target or overdosing of nearby sensitive tissue.

The treatment volume or PTV is typically planned with an internal target volume (ITV) approach in order to compensate for shape and position changes of the target due to the heartbeat at a fixed breathing level (often using a breath-hold methodology) or with the help of respiratory gating to accommodate a certain breathing phase or amplitude. To extract the ITV, the heart motion envelope can be extracted from a cardiac 4D CT scan using deformable registration of each phase of the cardiac 4D CT scan to the planning (reference) CT scan. The resulting ITV can comprise a union of the CTVs at all phases of the cardiac 4D CT. Finally, the planning target volume (PTV) can be generated by adding a 3-5 mm margin to the ITV based on typical patient set-up errors. This approach can still lead to relatively large volumes, and that can compromise the dosing of nearby organs-at-risk.

The present teachings address such problems associated with patient motion. In particular, these teachings provide tools and methods for a general workflow that can leverage an individualized model of the patient and their corresponding expected motion. Such a patient motion model can be built from various imaging modalities such as 4D/5D CT/CBCT/MR/PET and fluoroscopy and their combination. In lieu of the foregoing, or in combination therewith, these teachings will also accommodate employing a mid or mean position of, for example, a cardiac target that can be calculated resulting in a single three-dimensional image data set that can be used for treatment planning and patient positioning. There is also no restriction if it is patient-specific or based on data from a cohort (e.g., to represent statistics about typical motion). Although the present examples reference a cardiac radio ablation application it will be appreciated that these teachings can also find application in other fields of radiation therapy including, for example, hypofractionated, stereotactic, and novel forms of radiation therapy such as FLASH, and mini-and micro-beam radiation therapy.

During treatment planning

4D/5D CT/CBCT/MR/PET or alternatively image sequence resolved with respect to (in this example) both respiratory and cardiac motion, including CTV, and other structures of interest can be loaded to a viewer. These images can be user-segmented and/or auto-segmented to identify the clinical target volume (CTV) and organs-at-risk and register those features to all phases of the 4D/5D CT/CBCT/MR content.

These teachings will accommodate playing a combined representation of respiratory and cardiac motion. That said, per these teachings the user can selectively freeze (or slow) respiratory motion to thereby play only cardiac motion (or to at least emphasize the latter) or, alternatively, freeze (or slow) cardiac motion to thereby play only respiratory motion (or to at least emphasize the latter).

If desired, these teachings will support providing a display ruler to overlay on the viewer to facilitate measuring displacement manually. As desired, additional geometric measurement tools can be provided to metricize such things as overlap (by volume or percentage) between structures and/or the distance at, for example, a closest point between two structures.

By one approach, these teachings will accommodate automated assessment of CTV displacement due to cardiac and respiratory motion and automated generation of the internal target volume (ITV) or mid or mean target position. This can include taking into account deformation fields as desired. By one approach, the user can be allowed to play the motion information to thereby visually verify that the CTV motion is within the ITV.

By one approach, these teachings will accommodate a planning organ-at-risk volume (PRV) margin calculator. A user can choose/select a particular displayed structure after which these teachings will accommodate an automated assessment of structure displacement due to cardiac and respiratory motion and automated generation of a corresponding PRV. Relevant motion can then be displayed/played for the user to verify that the structure motion is within that PRV.

By one approach, and based on a treatment plan and a motion model as described herein, these teachings will accommodate calculating a probabilistic model to assess the expected success rate to successfully deliver the plan and/or to meet certain specific criteria as desired. The latter can serve, for example, to assess various candidate motion mitigation strategies. This can comprise, at least in part, assessing the impact on the CTV, structure motion, margins, dose plan, organs-at-risk, and/or therapeutic outcome when applying one or more of a variety of techniques such as, but not limited to, breath-holding, respiratory gating, tracking, use of a ventilation device, ECG-gating, and/or ICD pacing, to note some potentially useful options.

Day of treatment

These teachings will support comparing day-of-simulation and day-of-treatment motion. In particular, such comparisons can serve, for example to validate one or more motion models. This can comprise, for example, a geometric comparison of expected motion trajectories to determine whether an observed day-of-treatment motion is covered by the motion model. In these regards, these teachings can be leveraged to determine whether the observed motion can be represented by a normal set of parameters of the patient motion model, to detect potential overfitting of the model given the day-of-treatment motion, and/or whether the model estimates, for example, a high likelihood of the day-of-treatment motion.

As another example in these regards, these teachings will accommodate comparison of the model spans (i.e., whether a simulation model and a day-of-treatment model can or are expressing/representing a same motion).

If necessary, or desired, these teachings will support adapting plan in view of day-of-treatment observed motion. This may comprise, for example, re-evaluation of previously defined requirements. In lieu of the foregoing or in combination therewith, ITV, PRV, and various respective priorities can be checked and/or a treatment dry run on the new data could be performed.

These teachings will also accommodate dose reconstruction with 4D/5D CBCT and/or a multi-dimensional motion model (such as a 5D motion model). This can comprise, for example, calculating a delivered dose when taking motion into consideration and comparing a planned dose versus a delivered dose. These teachings will also support using the updated motion model of the day to apply the scheduled plan on this motion as part of a dry run. The latter would allow checking the intended treatment with regard to a dose volume parameter and then deciding if the scheduled plan can be executed in view of the observed motion of the day. If not, then an on-line plan adaptation could be triggered to help ensure that the intended treatment for this fraction of treatment is fulfilled. For fractionated treatments, these teachings can serve to facilitate compensating for underdosing/overdosing in future fractions. For single fraction plans, these teachings can facilitate compensating for underdosing immediately following a treatment exposure (that is, while the patient is still on the patient support platform in the radiation treatment platform and in between subsequent arcs). These teachings will also support real-time monitoring of organ-at-risk doses with an option to halt treatment early (i.e., prior to a planned conclusion of the treatment session) to protect a given organ-at-risk.

If desired, these teachings will serve to enable the use of an adaptive/dynamic ITV. For example, the treatment could be defined using the ITV as an upper bound. The corresponding margin could then be dynamically reduced (or increased) based on, for example, a current breathing pattern. Presuming, by way of example, the availability of multimodality 2D/3D/4D/5D planning images from the treatment simulation phase, temporal image sequences resolved with respect to respiratory and cardiac motion, (auto-)segmentations of a relevant target and organs-at-risk, and a patient motion model that is based on planning images (though it will be appreciated that multiple motion models could be generated, e.g., a motion model for the heart on the one hand and for respiratory activity on the other), the teachings will accommodate providing/using multiple radiation treatment plans including treatment strategies such as breath-hold, gating, tracking, ITV approach, plan based on mid or mean-position CT or CBCT, as well as back-up plans in case a particular respiratory controlled treatment strategy might not be feasible on the day of treatment. Several motion mitigation strategies based on the impact of CTV, ITV, PRV, OAR motion, and their margins can be used, such as but not limited to: Breath-hold, gating, beam or couch tracking, ventilation device, ECG-gating, ICD pacing, and so forth.

By one approach, an updated motion model (or models) of the day-of-treatment are presented to the user together with the original patient motion model(s) to help guide the decision-making and potential adaptive therapy workflow. Useful examples in these regards include (automatic) registration of both a previous model and a day-of-treatment model (or of multiple motion models in cases where multiple motion models from previous fractions are used), side-by-side dynamic visualization of user-selected motion models, and/or one or more blended views of dynamic motion models, including, if desired, a slider or other user interface to allow the user to fade in/out a given one of the models from the presentation.

By one approach, these teachings will facilitate using an overlay capability of target and organ-at-risk segmentations of the original and/or any adapted segmentation set. This may comprise, for example, use of motion model registrations to create (auto-)segmentations on a new motion model, use of automatic segmentation algorithms to create segmentations, automated (though user-defined if desired) analyses of center of mass (COM) motion differences (between, for example, targets and organs-at-risk), surface distances between segments, overlap between segments with respect to OAR/PRV/GTV/CTV/target to PTV margins used, and concerning the expected dose distribution, or any additional metric that is of clinical value (such as, for example, a distance to the closest points between structures). These teachings will also accommodate use of artificial intelligence-guided editing tools to adapt existing target and organ-at-risk segmentation to the patient motion model of the day-of-treatment.

These teachings will also accommodate enabling an overlay capability of the dose distribution of the day-of-treatment to a new (or updated) motion model. In these regards, these teachings will support, for example, use of motion model registrations to morph one or multiple pre-treatment three-dimensional dose distributions on the motion model of the day-of-treatment, an automated analyses of plan quality metrics and dose-volume thresholds defined for this patient in earlier phases, and/or an automated analyses of outcome-related measures based on the treatment plan and actual patient-based motion on the day-of-treatment.

When a treatment plan has been created using motion-robust planning techniques as described herein, it can be of interest to assess the similarity of the motion components. These teachings can serve to support analyzing the motion model of the day-of-treatment in terms of motion-robust planning requirements and/or a need to inform the user of any deviation or potential improvement in terms of robust dose planning.

By one approach, a motion model can be updated during treatment based on external inputs (such as kV images, respiration signals, patient surface monitoring, ECG, and so forth) and the updated motion model can then be used for real-time dose calculations. The latter, in turn, can enable dose tracking during treatment.

For example, the foregoing can include a real-time comparison regarding patient motion concerning expected motion and the ITV and PRV based on the expected motion. If the motion during treatment changes, a new dose plan can be adapted on the fly (or after a beam hold) to adapt to the changed motion of the anatomy.

Other examples in these regards include real-time comparisons of planned versus delivered dose during treatment for targets and/or organs-at-risk, controlling adjustable thresholds to initiate beam-hold if the deviation is above, for example, a user or clinic acceptable threshold, compensating for a dose deviation in a given fraction by adapting the plan for a planned follow-on fraction, and/or for single fraction treatments and during treatment, facilitating adaptations either after a beam-hold and re-analyses or based on close to real-time automated re-planning.

Such motion models can also be used for motion-adapted treatment delivery beyond beam-gating or tracking. Possible ways of taking motion during treatment delivery into account include, but are not limited to, slowing down and/or accelerating treatment delivery based on motion, delivering treatment only at those gantry angles where motion is minimally seen from the beams' eye view, selecting from amongst pre-computed plans as a function, at least in part, of the observed motion, and/or planning in some other way for expected motion.

Those skilled in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

What is claimed is:

1. A method to facilitate compensating for motion during a therapeutic treatment session for a particular patient, the method comprising:
by a control circuit:
accessing multi-dimensional information for the particular patient;
automatically determining a supplemental boundary for at least one portion of the particular patient as a function, at least in part, of the multi-dimensional information.

2. The method of claim 1, wherein the supplemental boundary comprises a margin that is added to a boundary of the at least one portion of the particular patient.

3. The method of claim 1, wherein the at least one portion of the particular patient comprises at least one of:
a treatment target;
an organ-at-risk.

4. The method of claim 1, further comprising:
determining a planning treatment volume as a function, at least in part, of the supplemental boundary.

5. The method of claim 1, wherein the multi-dimensional information includes motion-based imagery comprising at least two of cardiac-based imagery for the particular patient, respiratory-based imagery for the particular patient, and cyclic gastric motion-based imagery for the particular patient.

6. The method of claim 5, further comprising:
presenting the motion-based imagery to a user;
providing the user, via a user interface, with an opportunity to selectively modify movement of one motion-based imagery separately from another motion-based imagery.

7. The method of claim 1, further comprising:
generating a motion model for the particular patient as a function, at least in part, of the multi-dimensional information for the particular patient.

8. The method of claim 7, further comprising:
assessing efficacy for each of a plurality of different therapeutic treatment modalities for the particular patient as a function, at least in part, of the motion model.

9. The method of claim 7, further comprising:
accessing supplemental multi-dimensional information for the particular patient at a time of treatment;
updating the motion model as a function, at least in part, of the supplemental multi-dimensional information.

10. The method of claim 7, further comprising:
accessing supplemental multi-dimensional information for the particular patient at a time of treatment;
validating the motion model as a function, at least in part, of the supplemental multi-dimensional information

11. The method of claim 1, further comprising:
reconstructing an absorbed dose administered during the therapeutic treatment session as a function, at least in part, of at least one of:
the multi-dimensional information for the particular patient; and
a motion model for the particular patient that was generated as a function, at least in part, of the multi-dimensional information for the particular patient.

12. The method of claim 1, further comprising:
optimizing a radiation-based therapeutic treatment plan for the particular patient as a function of at least two different dimensions of movement as derived, at least in part, from the multi-dimensional information.

13. An apparatus to facilitate compensating for motion during a therapeutic treatment session for a particular patient, the apparatus comprising:
a control circuit configured and arranged to:
access multi-dimensional information for the particular patient;
automatically determine a supplemental boundary for at least one portion of the particular patient as a function, at least in part, of the multi-dimensional information.

14. The apparatus of claim 13, wherein the supplemental boundary comprises a margin that is added to a boundary of the at least one portion of the particular patient.

15. The apparatus of claim 13, wherein the at least one portion of the particular patient comprises at least one of:
a treatment target;
an organ-at-risk.

16. The apparatus of claim 13, wherein the control circuit is configured to:
automatically determine a planning treatment volume as a function, at least in part, of the supplemental boundary.

17. The apparatus of claim 13, wherein the multi-dimensional information includes motion-based imagery comprising at least two of cardiac-based imagery for the particular patient, respiratory-based imagery for the particular patient, and cyclic gastric motion-based imagery for the particular patient.

18. The apparatus of claim 17, wherein the control circuit is further configured to:
present the cardiac-based imagery and the respiratory-based imagery to a user;
provide the user, via a user interface, with an opportunity to selectively modify movement of one motion-based imagery separately from another motion-based imagery.

19. The apparatus of claim 13, wherein the control circuit is further configured to:
generate a motion model for the particular patient as a function, at least in part, of the multi-dimensional information for the particular patient.

20. The apparatus of claim 19, wherein the control circuit is further configured to:
assess efficacy for each of a plurality of different therapeutic treatment modalities for the particular patient as a function, at least in part, of the motion model.

21. The apparatus of claim 19, wherein the control circuit is configured to:
access supplemental multi-dimensional information for the particular patient at a time of treatment;
update the motion model as a function, at least in part, of the supplemental multi-dimensional information.

22. The apparatus of claim 19, wherein the control circuit is configured to:
access supplemental multi-dimensional information for the particular patient at a time of treatment;
validate the motion model as a function, at least in part, of the supplemental multi-dimensional information.

23. The apparatus of claim 13, wherein the control circuit is configured to:
reconstruct an absorbed dose administered during the therapeutic treatment session as a function, at least in part, of at least one of:
the multi-dimensional information for the particular patient; and
a motion model for the particular patient that was generated as a function, at least in part, of the multi-dimensional information for the particular patient.

24. The apparatus of claim 13, wherein the control circuit is further configured to:
optimize a radiation-based therapeutic treatment plan for the particular patient as a function of at least two different dimensions of movement as derived, at least in part, from the multi-dimensional information.
